(19) United States
(12) Patent Application Publication
(10) Pub. No.: US 2012/0142017 A1
(43) Pub. Date: Jun. 7, 2012

(54) BIOSENSOR DEVICE AND MANUFACTURING METHOD THEREOF

(75) Inventors: Chan-Woo PARK, Daejeon (KR); Jong-Heon Yang, Daejeon (KR); Chil-Seong Ah, Daejeon (KR); Wan-Joong Kim, Goyang (KR); Chang-Geun Ahn, Daejeon (KR); Gun-Yong Sung, Daejeon (KR)

(73) Assignee: Electronics and Telecommunications Research Institute, Daejeon (KR)

(21) Appl. No.: 13/289,424

(22) Filed: Nov. 4, 2011

(30) Foreign Application Priority Data
Dec. 6, 2010 (KR) 10-2010-0123542

(51) Int. Cl. G01N 33/53; C12M 1/34; G01N 33/82

(52) U.S. Cl. 435/7.1; 422/69; 436/501; 435/288.7

(57) ABSTRACT

Disclosed is a biosensor device, comprising: a capillary tube with probe molecules immobilized on the inner wall surface thereof, and a liquid sample containing target molecules, said biosensor device being characterized in that a contact angle between the inner wall surface of the capillary tube and the liquid sample changes because of the specific interaction between the probe molecules and the target molecules, which leads, in turn, to a change in the height of the liquid sample in the capillary tube.

### CROSS REFERENCE TO RELATED APPLICATION

This application claims the benefit of Korean Patent Application No. 10-2010-0123542, filed Dec. 6, 2010, which is hereby incorporated by reference in its entirety into this application.

### BACKGROUND OF THE INVENTION

1. Technical Field

The present invention relates to a biosensor device useful for quantitatively determining a target molecule in a liquid sample with the naked eye, which is based on the fact that a contact angle between the inner wall surface of the capillary tube and the liquid sample changes because of the specific interaction of the probe molecules with the target molecules, which leads, in turn, to a change in the height of the liquid sample in the capillary tube. Also, the present invention is concerned with a method for manufacturing the same.

2. Description of the Related Art

Most biosensors for detecting specific target biomolecules (proteins, enzymes, DNAs, etc.) in liquid biosamples have probe molecules immobilized onto the surface of a sensing part. These probe molecules specifically bind to specific target molecules, which allows the selective detection of the specific target molecules.

On the whole, a biosensor, which is an analytical device for the quantitative detection of the interaction of probe molecules with target molecules, is designed to act in an optical or an electrical manner. Optical biosensors are based on optical changes by optical signals generated from luminescents, such as fluorescents, phosphorescents, colorants, etc., labeled to target molecules which are conjugated with probes immobilized onto the surface of a sensing part. In many electrical biosensors, on the other hand, a probe is immobilized onto the surface of a field effect transistor channel, which is a sensing part, and when a target molecule binds to the immobilized probe, a channel current change is generated by the target molecule charge.

In addition to sensing devices that transform the signal resulting from the interaction of the target molecules with the probe molecules into an optical or electrical signal, these conventional biosensors require other analysis devices to detect and measure the transformed signals. In an optical biosensor, for example, an expensive optical system such as an optical scanner is employed to detect the signal of a luminescent. Many electrical biosensors require an instrument for measuring the microcurrent changes of ones to tens of nA at a high signal-to-noise ratio.

That is to say, conventional biosensors require a reader equipped with modules for detecting, processing and displaying sensor signals in addition to a sensing device. Since such a reader is difficult to embody into a cheap, portable system, conventional biosensors are problematic in terms of the user's convenience or accessibility, the rapidity of diagnosis, and cost.

### SUMMARY OF THE INVENTION

Accordingly, the present invention has been made keeping in mind the above problems occurring in the prior art, and an object of the present invention is to provide a novel biosensor device and method for quantitatively analyzing a target molecule in a biosample in situ without additional analysis devices, but with the naked eye.

In order to accomplish the above object, the present invention provides a biosensor device, comprising, a capillary tube with probe molecules immobilized on the inner wall surface thereof; and a liquid sample containing target molecules filled in the capillary tube, said biosensor device being characterized in that a contact angle between the inner wall surface of the capillary tube and the liquid sample changes because of the specific interaction of the probe molecules with the target molecules, which leads, in turn, to a change in the height of the liquid sample in the capillary tube.

Also, the present invention provides a method for manufacturing a biosensor device, comprising:

preparing a capillary tube;

immobilizing probe molecules onto the inner wall surface of the capillary tube;

preparing a liquid sample containing target molecules capable of binding specifically to the probe molecules; and

inserting the capillary tube in the liquid sample.

Without employing a reader, the biosensor device of the present invention comprises only a sensor element that makes it possible to determine the quantity of a target molecule in a liquid biosample with the naked eye. Accordingly, the biosensor device allows the user to quantitatively analyze a target molecule in situ with promptness and convenience, and enjoys the advantage of cutting back on the expenses incurred in the manufacture and operation of the reader.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a schematic view showing a contact angle (Qc) between a flat solid surface and a liquid sample drop placed on the surface.

FIG. 2 is a schematic view showing a change in contact angle between a substrate and a liquid sample containing streptavidin as the streptavidin binds to biotin molecules immobilized on the surface of the substrate.

FIG. 3 is a schematic view showing the ascending or descending of the level of liquid samples in capillary tubes according to capillary pressure.

FIG. 4 is a schematic view showing a change in the height of a liquid sample within a capillary tube as the contact angle is decreased by the interaction of the target molecules (θc1>θc2) or as the concentration of the target molecules is shifted from a lower level (h1) to a higher level (h2).

### DESCRIPTION OF THE PREFERRED EMBODIMENTS

Reference now should be made to the drawings, in which the same reference numerals are used throughout the different drawings to designate the same or similar components.

The biosensor device according to the present invention comprises a capillary tube with probe molecules immobilized on the inner wall surface thereof; and a liquid sample containing target molecules filled in the capillary tube, characterized in that a contact angle between the inner wall surface of the capillary tube and the liquid sample changes because of the specific interaction between the probe molecules and the target molecules, which leads, in turn, to a change in the height of the liquid sample in the capillary tube.

As used herein, the term “capillary tube” refers a tube which is thin and long enough to allow a capillary phenomenon.

The capillary phenomenon is a phenomenon whereby when a capillary tube is inserted in a liquid, the level of the liquid in the capillary rises or falls because of the combined effect of the cohesion of the liquid and the adhesion between the liquid and the capillary tube.

Preferably, the capillary tube used in the present invention is transparent so that the height of the liquid sample within the capillary tube is visible with the naked eye.

On the surface of the outer wall of the capillary tube, scales may be marked to allow the height of the liquid sample to be readily determined.

The probe molecule useful in the present invention is one designed to specifically bind to a target molecule of interest. It may be a protein, an enzyme, DNA or the like. A non-limiting, illustrative preferred example is biotin.

The target molecule shows specific interaction with the probe molecule and may be a protein, an enzyme, DNA, etc. Preferred is streptavidin, but this is a non-limiting example.

So long as the density of the liquid sample used in the present invention is large enough to guarantee the capillary phenomenon, no particular limitations are imparted thereto.

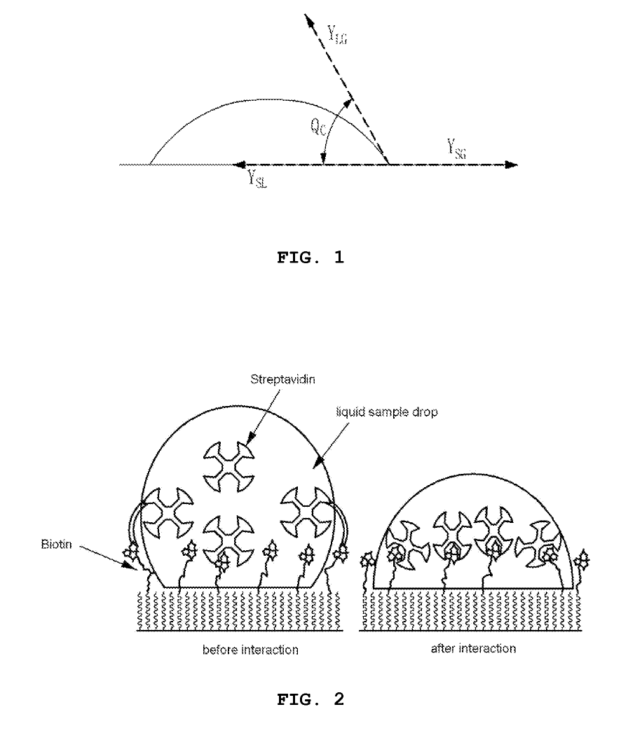

The contact angle (Qc) between a flat solid surface and a liquid sample drop placed thereon is defined as shown in FIG. 1 and calculated according to the following Math Equation 1:

$$cos {θ}_{c} = \frac{{γ}_{SG} - {γ}_{SL}}{{γ}_{LG}} \qquad [ Math Equation 1 ]$$

wherein γ_{LG}, γ_{SL} and γ_{SG} are respectively interfacial energies between liquid and gas, between solid and liquid and between a solid and a gas.

As is apparent from the equation, the contact angle (Qc) between the liquid sample and the solid surface decreases with the decrease in the solid-liquid interfacial energy (γ_{SL}).

After dropping a liquid sample containing a target molecule streptavidin on a glass substrate to the substrate of which the probe molecule biotin was immobilized, Rio and Smirnov monitored the contact angle between the liquid sample and the glass substrate over time, and identified that the contact angle gradually decreased with an increase in the streptavidin level or with the extension of the reaction time (Applied Materials and Interfaces Vol 1, No. 4, 768-774 (2009)).

As illustrated in FIG. 2, the interfacial energy (γ_{SL}) between the solid substrate and the liquid sample decreases as the streptavidin binds to the biotin molecules immobilized to the surface of the substrate. The decline of the interfacial energy (γ_{SL}) was found to increase with the increase of the streptavidin level in the sample. These results indicate that the specific interaction of biotin with streptavidin at the interface between the solid substrate and the liquid sample brings about a change in the interfacial energy, thus altering the contact angle.

The biosensor device of the present invention is designed to take advantage of the capillary phenomenon to visibly determine the change of contact angle with the number of target molecules in a sample.

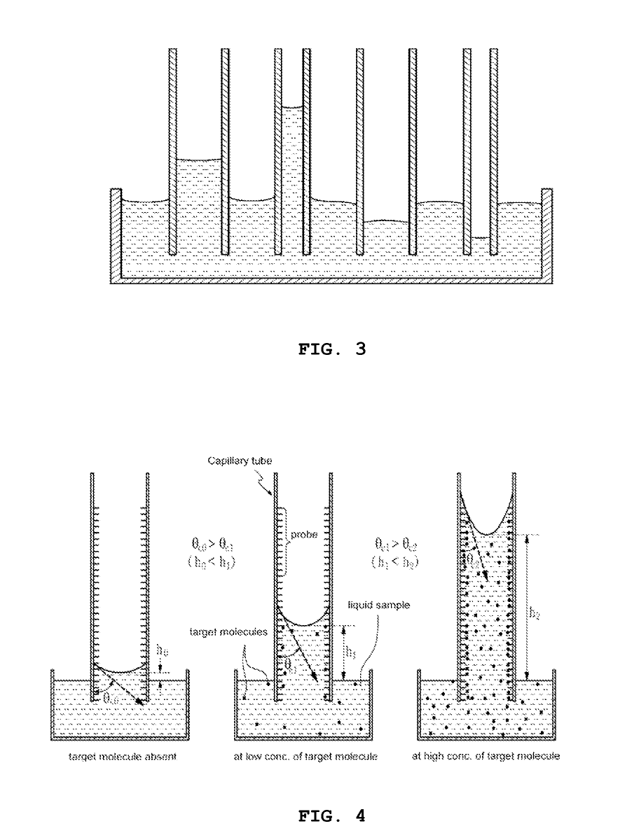

When a capillary tube with a sufficiently small radius and with probe molecules immobilized to the inner wall thereof is inserted into a liquid sample, as illustrated in FIG. 3, the liquid level within the capillary tube goes up or down because of capillary pressure generated between the menisci that are formed within the capillary tubes. The height of the liquid sample is given by the following Math Equation 2:

$$h = \frac{2 {γ}_{LG} cos {θ}_{c}}{ρ g r} \qquad [ Math Equation 2 ]$$

wherein h represents the height of the liquid sample, γ_{LG} is a liquid-gas interfacial energy, θc is the contact angle, ρ is the density of liquid sample, g is gravity acceleration, and r is radius of the capillary tube.

That is, given that the liquid-gas interfacial energy (γ_{LG}) is constant, the h is positive (ascendant) when θc is less than 90° and negative (descendent) when θc is greater than 90°.

Therefore, as described above, the contact angle (θc) between the liquid sample and the inner wall of the capillary tube changes because of the interaction of the target molecules with the probe, which leads to an alternation in the height of the liquid sample within the capillary tube. In addition, the degree of change of the contact angle and thus the height is in proportion to the number of the target molecules binding to the probe immobilized to the inner wall of the capillary tube, so that it may be used as an index for the concentration of target molecules in the sample.

Based on this principle, the biosensor device of the present invention comprising a capillary tube with probe molecules immobilized on the inner wall surface thereof, and a liquid sample containing target molecules is operated in such a way that after the transparent capillary tube has been inserted for a predetermined period of time, the height of the liquid sample in the capillary tube is measured and compared with the normalized one to quantitatively determine the concentration of the target molecule in the liquid sample.

Also, the present invention provides a method for manufacturing a biosensor device, comprising:

preparing a capillary tube;

immobilizing probe molecules onto the inner wall surface of the capillary tube;

preparing a liquid sample containing target molecules capable of binding specifically to the probe molecules; and

inserting the capillary tube in the liquid sample.

Preferably, the capillary tube useful in the present invention is transparent so that the height of the liquid sample within the capillary tube is visible with the naked eye. On the surface of the outer wall of the capillary tube, scales may be marked to allow the height of the liquid sample to be readily determined.

According to the present invention, the user can determine the quantity of a target molecule of interest in a biosample in situ with the naked eye.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A biosensor device, comprising:
a capillary tube with probe molecules immobilized on the inner wall surface thereof; and
a liquid sample containing target molecules filled in the capillary tube,
characterized in that a contact angle between the inner wall surface of the capillary tube and the liquid sample changes because of the specific interaction between the probe molecules and the target molecules, which leads, in turn, to a change in the height of the liquid sample in the capillary tube.

2. The biosensor device of claim 1, wherein the capillary tube is transparent, with a scale marked on an outer wall thereof.

3. The biosensor device of claim 1, wherein the probe molecule is a protein, an enzyme or a DNA, and the target molecule is a protein, an enzyme or a DNA.

4. The biosensor device of claim 1, wherein the probe molecule is biotin and the target molecule is streptavidin.

5. The biosensor device of claim 1, wherein the contact angle is changed according to the number of the target molecules in the liquid sample.

6. The biosensor device of claim 1, wherein the height of the liquid sample changes in proportion to the number of the target molecules binding to the probe molecules immobilized to the inner wall of the capillary tube.

7. The biosensor device of claim 1, wherein the height of the liquid sample within the capillary tube is represented by the following Math Equation 2:
$$h = \frac{2 {γ}_{LG} cos {θ}_{c}}{ρ g r} \qquad [ Math Equation 2 ]$$
wherein h represents the height of the liquid sample, γ_{LG} is a liquid-gas interfacial energy, θc is the contact angle between the liquid sample and the solid surface, ρ is density of the liquid sample, g is gravity acceleration, and r is radius of the capillary tube, and
given that the liquid-gas interfacial energy (γ_{LG}) is constant, the h is positive (ascendant) when θc is less than 90° or less and negative (descendant) when θc is greater than 90°.

8. The biosensor device of claim 1, wherein the device is operated in such a way that after the transparent capillary tube has been inserted for a predetermined period of time, the height of the liquid sample in the capillary tube is measured and compared with a normalized one to quantitatively determine the concentration of the target molecules in the liquid sample.

9. A method for manufacturing a biosensor device, comprising:
preparing a capillary tube;
immobilizing probe molecules onto the inner wall surface of the capillary tube;
preparing a liquid sample containing target molecules capable of binding specifically to the probe molecules; and
inserting the capillary tube in the liquid sample.

10. The method of claim 9, wherein the capillary tube is transparent, with a scale marked on an outer wall thereof.

11. The method of claim 9, wherein the probe molecule is a protein, an enzyme or a DNA, and the target molecule is a protein, an enzyme or a DNA.

12. The method of claim 9, wherein the probe molecule is biotin and the target molecule is streptavidin.
